(19) United States
(12) Patent Application Publication
(10) Pub. No.: US 2024/0390476 A1
(43) Pub. Date: Nov. 28, 2024

(54) ATTENUATED ALPHAVIRUS

(71) Applicant: Zoetis Services LLC, Parsippany, NJ (US)

(72) Inventors: Elin PETTERSON, Oslo (NO); Ida LIEUNGH, Oslo (NO); Charlotte Andrea Hauge HANDELAND, Oslo (NO); Inge SOLBAKK, Oslo (NO); Espen HAALAND, Baerums Verk (NO); Anette FUREVIK, Askoy (NO); Trygve Meum ELIASSEN, Kolbotn (NO)

(21) Appl. No.: 18/686,637

(22) PCT Filed: Aug. 30, 2022
(86) PCT No.: PCT/US2022/075674

§ 371 Date: Feb. 26, 2024

Related U.S. Application Data
(60) Provisional application No. 63/238,812, filed on Aug. 31, 2021.

(51) Int. Cl. A61K 39/12; A61K 39/00; A61P 31/14; C07K 14/005; C12N 7/00; C12Q 1/70

(52) U.S. Cl. CPC A61K 39/12; A61P 31/14; C07K 14/005; C12N 7/00; C12Q 1/701; A61K 2039/5254; A61K 2039/552; C12N 2770/36121; C12N 2770/36122; C12N 2770/36134

(57) ABSTRACT

Disclosed is a mutated E2 protein of Salmonid alphavirus. An attenuated Salmonid Alphavirus comprising this protein as well as the methods of using and methods of detecting said attenuated virus are also disclosed.

### FIELD OF THE INVENTION

This invention is in generally in the field of attenuated alphaviruses and vaccines comprising the same.

### BACKGROUND

Pancreatic Disease (PD) is a serious disease that affects fish, in particular salmonid fish such as Atlantic salmon, rainbow trout and the like. The disease causes lesions in the pancreas, including loss of pancreatic exocrine tissue, and fibrosis, cardiac and skeletal muscle myopathies.

The causative agent of PD in salmon and rainbow trout is Salmon Pancreas Disease Virus (SPDV), commonly known as salmonid alphavirus (SAV). Based on sequence data of the SAV E2 structural protein and the non-structural protein 3 (nsP3), SAV strains can be assigned to at least six different subtypes: SAV-1, SAV-2, SAV-3, SAV-4, SAV-5 and SAV-6).

Norwegian PD outbreaks have been mainly caused by SAV-3, with the remaining subtypes occurring in the British Isles. However, SAV-2 outbreaks have also recently been detected in Norwegian salmon populations. Horizontal transmission of PD has been demonstrated and is believed to be the predominant transmission route, supported by the extended survival of virus in seawater. The virus is likely endemic in historically infected areas, based on evidence that outbreaks have been shown to recur in successive generations of salmon introduced on sites despite extensive fallow periods. In support of speculations that a substantial infection reservoir might exist in the seawater environment.

Pancreas disease caused by SAV2 and SAV3 leads to significant economic losses in the Norwegian salmonid production. Based on a stochastic model, Aunsmo and co-workers estimated that PD increased the production cost by 0.72 € per kg (Aunsmo, et al, Prev Vet Med 93(2-3): 233-41 (2010). In a more recent publication, the cost of a SAV3 outbreak on a farm with 1,000,000 smolt was estimated to 7.1 million € (Pettersen et al, Prev Vet Med 121(3-4):314-24 (2015)). The increased production cost is due to increased mortality, reduced growth, feed conversion and carcass quality (Bang Jensen, et al Dis Aquat Organ. 102(1):23-31 (2012), Larsson et al., Aquaculture 330-333 (2012) 82-91; Lerfall et al., Aquaculture 324-325 (2012) 209-217. Impaired growth is assumedly caused by reduced feed intake and impaired feed digestion.

Moriette et al refer to an attenuated salmonid alpha virus comprising mutations the 6K and E1 proteins in addition to E2. See J Virol., 80(8): 4088-98 (2006). Moriette reports that the virulent phenotype of Sleeping Disease Virus (SDV, freshwater SAV-2) was essentially associated with two amino acid changes, V8A and M136T, in the E2 glycoprotein, with the V8A change mostly being involved in the acquisition of the virulent phenotype.

### SUMMARY OF THE INVENTION

In the first aspect, the invention provides an amino acid sequence that is 94% identical to SEQ ID NO: 1, wherein an amino acid at position 233 is not threonine or is absent. Preferably, an amino acid at position 90 is not asparagine.

In the second aspect, the invention provides a nucleic acid sequence encoding the amino acid sequence according to any of the embodiments according to the first aspect. In certain embodiments, the nucleic acid sequence is SEQ ID NO: 3.

In the third aspect, the invention provides a vector comprising the nucleic acid sequence according to the second aspect of the invention.

In the fourth aspect, the invention comprises a host cell comprising the nucleic acid according to the second aspect of the invention.

In the fifth aspect, the invention provides an alphavirus comprising the amino acid sequence according to the first aspect of the invention. In different embodiments, the alphavirus is SAV-1, SAV-2, SAV-3, SAV-4, SAV-5, or SAV-6.

In the sixth aspect, the invention provides a vaccine comprising the virus according to any of the embodiments of the fifth aspect of the invention.

In the seventh aspect, the invention provides a method of using the vaccine to elicit a protective immune response against an alphavirus. In certain embodiments, the alphavirus is SAV-1, SAV-2, SAV-3, SAV-4, SAV-5 or SAV-6.

In the eighth aspect, the invention provides a method of determining whether a sample contains a nucleic acid sequence encoding the amino acid sequence according to any of the embodiments of the first aspect of the invention, the method comprising contacting said sample with a primer or a probe, wherein
a) said primer or probe differentially hybridizes to the nucleic acid sequence encoding at least a portion of SEQ ID NO: 1 depending on whether an amino acid at position 90 of SEQ ID NO: 1 is asparagine encoded by codon aac; and/or
b) said primer or probe differentially hybridizes to the nucleic acid sequence encoding at least a portion of SEQ ID NO: 1 depending on whether an amino acid at position 233 of SEQ ID NO: 1 is threonine; and/or
c) said primer or probe differentially hybridizes to the nucleic acid sequence encoding at least a portion of SEQ ID NO: 1 depending on whether an amino acid at position 375 of SEQ ID NO: 1 is threonine encoded by codon acc.

### DETAILED DESCRIPTION

The term “about” as applied to a reference number refers to the reference number plus or minus 10 of said value.

Amino acid numbering is provided according to the reference sequence. Thus, for example, in an amino acid sequence comprising SEQ ID NO: 1, position 90 corresponds to the amino acid at position 90 of SEQ ID NO: 1, once the amino acid sequence comprising SEQ ID NO: 1 is aligned to SEQ ID NO: 1 itself to achieve maximum alignment.

The term “attenuated” refers to the reduced ability of the virus to cause symptoms of an infection. The attenuation may be determined in different ways.

In certain embodiments, attenuation may be determined based on characteristics of the fish. For example, Pettersen et al (2015) summarized the biological effects associated with a pancreas disease (SAV-3) outbreak on a salmon farm with 1,000,000 smolts based on the average of the expert panel's weighted estimates.

The minimal effects of the disease depend on the age (weight) of the fish and are provided in Table 1. See Pettersen et al, 2015 (Preventive Veterinary Medicine 121 (2015) 314-324).

TABLE 1
Minimum and most likely effects of SAV-3 outbreak

| | Before day 319 | | After day 319 | |
|---|---|---|---|---|
| | (under 2.5 kg) | | (over 2.5 kg) | |
| Metrics | Minimum | Most Likely | Most likely | Baseline |
| Biologic Feed | 0.04 | 0.11 | 0.10 | 1.185 |
| Conversion ratio | | | | |
| Ordinary quality (%) | 0.66 | 2.66 | 3.13 | 2.72 |
| Production quality (%) | 1.41 | 2.21 | 2.57 | 2.25 |
| Condemned quality (%) | 0.74 | 1.87 | 1.99 | 0.68 |
| Mortality (%) | 2.87 | 8.11 | 7.61 | 16.3 |
| Growth reduction (kg) | 0.46 | 1.09 | 1.17 | 0 |

The biological effects are specified as the difference from the baseline scenario

The presence of the disease may be diagnosed by the combination of weight loss and the presence of histopathological lesions in pancreas and heart, which are the target organs of the virus, in combination with weight loss. Thus, in certain embodiments, the attenuated virus induces only minimal or no damage to heart or pancreas of the fish and/or the attenuated virus induces weight loss which is less than 90% of the weight reduction caused by the non-attenuated virus, and can be less than 80%, less than 70%, less than 60%, less than 50%, less than 40%, less than 30%, less than 20% or less than 10% of the most likely effect of the non-attenuated virus on the weight loss in the fish of the corresponding age (weight).

Attenuation may be determined based on the damage (or lack of damage) to pancreas, heart, or skeletal muscle. The damage to these organs may be scored as provided in Table 2 below:

TABLE 2
Organ damage scoring criteria.

| Scores | Heart Ventricle | Pancreas | Skeletal muscle |
|---|---|---|---|
| 0-1.0 | Focal necrosis of myocytes | Necrosis / apoptosis of individual | Focal (<3 foci) necrosis |
| | (individual cells), 5-10 foci/cells | cells with/without focal infiltration | of white muscle tissue |
| | in each specimen (apoptotic cells). | of inflammatory cells around | and/or red muscle. |
| | Subacute/chronic: Focal/multifocal | vascular structures and pancreatic | Usually no |
| | inflammatory changes (1-2 foci per | ducts. Few (1-5) foci with | inflammation |
| | field) including a few inflammatory | necrosis. | |
| | cells (<5 per focus) with or without | | |
| | necrosis. | | |
| 1.1-2.0 | Acute: Multifocal necrosis up to 10 | Focal to diffuse infiltration of | Multifocal necrosis (>3) |
| | per field at 20x magnification. | inflammatory cells around | of white muscle tissue |
| | Necrosis may be accompanied by | vascular structures and | and/or involvement of |
| | infiltration of inflammatory cells, | pancreatic ducts. Exocrine tissue | red muscle with focal to |
| | but usually mild and with few cells. | present but with necrosis and | multifocal distribution |
| | Subacute to chronic: Multifocal in- | areas of loss of structure/cellular | of necrosis and/or |
| | flammatory changes (>3 and <10 foci | integrity. | inflammation. |
| | per field) with/without necrosis, with | | |
| | >5 inflammatory cells per focus, | | |
| | areas with diffuse inflammation. | | |
| 2.1-3.0 | Acute: Multifocal necrosis >10 per | Infiltration of lymphocytic cells | Multifocal necrosis (>5) |
| | field at 20x magnification | varies, typically focal to scattered. | of white muscle tissue |
| | accompanied by moderate to strong | Loss of exocrine pancreas can go | and/or red muscle. |
| | infiltration of inflammatory cells. | without inflammation. Lesser | Multifocal to diffuse |
| | Distinct endothelial hyperplasia. | exocrine pancreas present gives | inflammatory pattern. |
| | Subacute to chronic: Diffuse | higher scores. | Diffuse pattern score> |
| | inflammatory changes with/without | Just remnants (islets) of exocrine | |
| | necrosis (score > 2.5). | pancreatic tissue (score >2.5). | |
| | | No intact exocrine tissue | |
| | | with/without inflammation | |
| | | (score = 3). | |

Thus, in certain embodiments, the attenuated virus according to the invention may not be transmitted from a vaccinated fish to a naïve fish.

In the most preferred embodiments, the attenuated virus according to the invention does not cause damage above score 2.0 in any of the organs recited in Table 2. (The grading scale follows Karlsen et al 2012 Vaccine 30 issue 38 p 5688-5694). Preferably, the attenuated virus according to the invention does not cause damage above score 1.0 in any of the organs recited in Table 2.

The phrase “conditions optimal for differential hybridization” reflects the fact that the intensity (or the probability) of hybridization between two nucleic acid molecules refers not only to the degree of complementarity but also on conditions of hybridization, including, for example, temperature and ionic strength of the solution. Conditions optimal for differential hybridization are such that one can differentiate the binding of two different primers or probes to the template, when said primers or probes differ by a single nucleotide. A person of ordinary skill in the art would appreciate that conditions optimal for differential hybridization include the length of the sequences, the degree of complementarity between the primer/probe and the template, the nature of the sequence (e.g., CG content), the temperature, the ionic strength of the solution and other factors. See, e.g., Current Protocols in Molecular Biology, John Wiley & Sons, N.Y. (1989), Kashima et al. (1985) Nature 313:402-404, and Sambrook et al. (1989) Molecular Cloning: A Laboratory Manual, 2nd Ed., Cold Spring Harbor Laboratory, Cold Spring Harbor, N.Y. (“Sambrook”); Haymes et al., “Nucleic Acid Hybridization: A Practical Approach”, IRL Press, Washington, D.C. (1985), Gibbs et al. (1989) Nucleic Acids Res. 17:2437-2448, Prossner (1993) Tibtech 11:238.

The phrase “differentially hybridizes” indicates that under conditions optimal for differential hybridization one can differentiate the binding of two different primers or probes to the template, when said primers or probes differ by a single nucleotide. The term also includes the ability of the primer to initiate amplification of the template. It is known that the amplification proceeds from the 5′ to the 3′. Thus, if there is a mismatch at the 3′ end of the primer, the amplification of the template is likely to be impaired compared to the primers which provide for a perfect complementarity to the template at the primer's 3′ end, preferably the terminal 3′ nucleotide. If, for example and without limitation, two 20-nucleotide long primers hybridize to the same portion of a template, but the first primer has two mismatches in the middle and the second primer has two mismatches at the two terminal 3′ bases, amplification initiated from the first primer is likely to be more efficient than the amplification initiated from the second primer. Thus, even though the first and the second primer exhibit the same percent complementarity to the template (90% for each), they still differentially hybridize to the template because the amplification initiated from the first primer is likely to result in a greater amount of the product than the amplification initiated from the second primer.

“Therapeutically effective amount” refers to an amount of an antigen or vaccine that would induce an immune response in a subject receiving the antigen orvaccine which is adequate to prevent or reduce signs or symptoms of disease, including adverse health effects or complications thereof, caused by infection with a pathogen, such as a virus or a bacterium. Humoral immunity or cell-mediated immunity or both humoral and cell-mediated immunity may be induced. The immunogenic response of an animal to a vaccine may be evaluated, e.g., indirectly through measurement of antibody titers, lymphocyte proliferation assays, or directly through monitoring signs and symptoms after challenge with wild type strain. The protective immunity conferred by a vaccine can be evaluated by measuring, e.g., reduction in clinical signs such as mortality, morbidity, temperature number, overall physical condition, and overall health and performance of the subject. The amount of a vaccine that is therapeutically effective may vary depending on the particular adjuvant used, the particular antigen used, or the condition of the subject, and can be determined by one skilled in the art.

“Treating” refers to preventing a disorder, condition, or disease to which such term applies, or to preventing or reducing one or more symptoms of such disorder, condition, or disease.

In a first aspect, the invention provides an amino acid sequence that is 94% identical to SEQ ID NO: 1, wherein an amino acid at position 233 is not threonine or is absent, and optionally, wherein an amino acid at position 90 is not asparagine.

Preferably, the amino acid at position 90 is neither asparagine nor glutamine. Even more preferably, the amino acid at position 90 is aspartic acid or glutamic acid.

The amino acid at position 233 is not threonine. In certain embodiments, the amino acid at position 233 is selected from the group consisting of the remaining nineteen L-amino acids that can be translated from DNA in fish cells. In other embodiments, the amino acid at position 233 of SEQ ID NO: 1 is absent.

In addition to these amino acid changes, the amino acid sequence of the invention may further comprise a substitution at amino acid 375. In naturally existing SEQ ID NO: 1, the amino acid at position 375 is threonine. Accordingly, in certain embodiments, the amino acid according to the invention contains an amino acid other than threonine at position 375. It is preferred that threonine at position 375 is substituted with isoleucine or leucine, with isoleucine being the most preferable substitution.

Earlier references disclose importance of N-terminal amino acids in E2 protein for virulent phenotype. For example, Merour et al discloses that virulent phenotype of Sleeping Disease Virus was associated with Alanine at position 8 and Threonine at position 362, with Alanine at position 8 being responsible for 90% of the virulence compared to an attenuated Sleeping Disease Virus (SDV) that contains Valine at position 8. See J Virol, 87(10): 6027-6030 (2013).

The amino acid sequence of the instant invention, in certain embodiments, contains Alanine at position 8 and still is highly attenuated. In other embodiments, the amino acid sequence of the invention contains Valine at position 8, and, optionally, methionine at position 365, in addition to the mutation at position 233 and, optionally, 90 and/or 375, as described above. The additional mutation at the position known to be important for virulence provides additional safeguard against reversion to virulence.

The amino acid sequences according to the invention are at least 94% identical to SEQ ID NO: 1, and contain different combinations of the mutations described above. Thus, in different embodiments, the amino acid sequence of the invention is at least 95% identical, or at least 96% identical or at least 97% identical, or at least 98% identical, or at least 99% identical to SEQ ID NO: 1. Thus, the amino acid sequence of the invention may differ from SEQ ID NO: 1 by 2, 3, 4, 5, 6, 7, 8, 9, 10 or more amino acids, including the mutation at position 233 and, optionally positions 90 and/or 375.

The differences between the protein of the invention and SEQ ID NO: 1, other than the specific mutations described above, may be due to insertions, deletions, or substitutions, or combinations thereof. In certain embodiments the differences are due to substitutions. In certain embodiments, the substitutions are conservative substitutions.

The skilled person will further acknowledge that alterations of the nucleic acid sequence resulting in modifications of the amino acid sequence of the protein it codes may have little, if any, effect on the resulting three-dimensional structure of the protein. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in the substitution of one negatively charged residue for another, such as aspartic acid for glutamic acid, or one positively charged residue for another, such as lysine for arginine, can also be expected to produce a protein with substantially the same functional activity.

The following six groups each contain amino acids that are typical conservative substitutions for one another: [1] Alanine (A), Serine (S), Threonine (T); [2] Aspartic acid (D), Glutamic acid (E); [3] Asparagine (N), Glutamine (Q); [4] Arginine (R), Lysine (K), Histidine (H); [5] Isoleucine (1), Leucine (L), Methionine (M), Valine (V); and [6] Phenylalanine (F), Tyrosine (Y), Tryptophan (W), (see, e.g., US Patent Publication 20100291549).

Protein and/or nucleic acid sequence identities can be evaluated using any of the variety of sequence comparison algorithms and programs known in the art. For sequence comparison, typically one sequence acts as a reference sequence (e.g., a sequence disclosed herein), to which test sequences are compared. A sequence comparison algorithm then calculates the percent sequence identities for the test sequences relative to the reference sequence, based on the program parameters.

The percent identity of two amino acid or two nucleic acid sequences can be determined for example by comparing sequence information using the computer program GAP, i.e., Genetics Computer Group (GCG; Madison, WI) Wisconsin package version 10.0 program, GAP (Devereux et al. (1984), Nucleic Acids Res. 12: 387-95). In calculating percent identity, the sequences being compared are typically aligned in a way that gives the largest match between the sequences. The preferred default parameters for the GAP program include: (1) The GCG implementation of a unary comparison matrix (containing a value of 1 for identities and 0 for non-identities) for nucleotides, and the weighted amino acid comparison matrix of Gribskov and Burgess, ((1986) Nucleic Acids Res. 14: 6745) as described in Atlas of Polypeptide Sequence and Structure, Schwartz and Dayhoff, eds., National Biomedical Research Foundation, pp. 353-358 (1979) or other comparable comparison matrices; (2) a penalty of 8 for each gap and an additional penalty of 2 for each symbol in each gap for amino acid sequences, or a penalty of 50 for each gap and an additional penalty of 3 for each symbol in each gap for nucleotide sequences; (3) no penalty for end gaps; and (4) no maximum penalty for long gaps.

Sequence identity and/or similarity can also be determined by using the local sequence identity algorithm of Smith and Waterman, 1981, Adv. Appl. Math. 2:482, the sequence identity alignment algorithm of Needleman and Wunsch, 1970, J. Mol. Biol. 48:443, the search for similarity method of Pearson and Lipman, 1988, Proc. Nat. Acad. Sci. U.S.A. 85:2444, computerized implementations of these algorithms (BESTFIT, FASTA, and TFASTA in the Wisconsin Genetics Software Package, Genetics Computer Group, 575 Science Drive, Madison, Wis.).

Another example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, 1987, J. Mol. Evol. 35:351-360; the method is similar to that described by Higgins and Sharp, 1989, CABIOS 5:151-153. Useful PILEUP parameters including a default gap weight of 3.00, a default gap length weight of 0.10, and weighted end gaps.

Another example of a useful algorithm is the BLAST algorithm, described in: Altschul et al., 1990, J. Mol. Biol. 215:403-410; Altschul et al., 1997, Nucleic Acids Res. 25:3389-3402; and Karin et al., 1993, Proc. Natl. Acad. Sci. U.S.A. 90:5873-5787. A particularly useful BLAST program is the WU-BLAST-2 program obtained from Altschul et al., 1996, Methods in Enzymology 266:460-480. WU-BLAST-2 uses several search parameters, most of which are set to the default values. The adjustable parameters are set with the following values: overlap span=1, overlap fraction=0.125, word threshold (T)=II. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., 1993, Nucl. Acids Res. 25:3389-3402. Gapped BLAST uses BLOSUM-62 substitution scores; threshold T parameter set to 9; the two-hit method to trigger ungapped extensions, charges gap lengths of k a cost of 10+k; Xᵤ set to 16, and X_{g} set to 40 for database search stage and to 67 for the output stage of the algorithms. Gapped alignments are triggered by a score corresponding to about 22 bits.

In certain embodiments, the amino acid sequence of the invention comprises SEQ ID NO: 2.

In the second aspect, the invention discloses a nucleic acid sequence encoding the amino acid sequence according to any of the embodiments according to the first aspect. In certain embodiments, the nucleic acid sequence is SEQ ID NO: 3. In certain embodiments, the nucleic acid sequence of the invention may be codon-optimized, depending on the needs of the person of ordinary skill in the art who is practicing the invention.

Techniques to obtain the polypeptides according to the invention are well known in the art. For example, genetic engineering techniques and recombinant DNA expression systems may be used.

Nucleic acid molecules encoding the amino acid sequences according to any embodiment of the first aspect of the invention may also be inserted into a vector (e.g., a recombinant vector) such as one or more non-viral and/or viral vectors. Non-viral vectors may include, for instance, plasmid vectors (e.g., compatible with bacterial, insect, and/or mammalian host cells). Exemplary vectors may include, for example, PCR-ii, PCR3, and pcDNA3.1 (Invitrogen, San Diego, Calif.), pBSii (Stratagene, La Jolla, Calif.), pet15 (Novagen, Madison, Wis.), pGEX (Pharmacia Biotech, Piscataway, N.J.), pEGFp-n2 (Clontech, Palo Alto, Calif.), pET1 (Bluebacii, Invitrogen), pDSR-alpha (PCT pub. No. WO 90/14363) and pFASTBACdual (Gibco-BRL, Grand island, NY) as well as Bluescript plasmid derivatives (a high copy number COLe1-based phagemid, Stratagene Cloning Systems, La Jolla, Calif.), PCR cloning plasmids designed for cloning TAQ-amplified PCR products (e.g., TOPO™ TA Cloning® kit, PCR2.1© plasmid derivatives, Invitrogen, Carlsbad, Calif.). Bacterial vectors may also be used including, for instance, Shigella, Vibrio cholerae, Lactobacillus, Bacille Calmette Guerin (BCG), and Streptococcus (see for example, WO 88/6626; WO 90/0594; WO 91/13157; WO 92/1796; and WO 92/21376). The vectors may be constructed using standard recombinant techniques widely available to one skilled in the art. Many other non-viral plasmid expression vectors and systems are known in the art and may be used.

In the third aspect, the invention provides a vector comprising the nucleic acid sequence according to the second aspect of the invention. Various viral vectors that have been successfully utilized for introducing a nucleic acid to a host include retrovirus, adenovirus, adeno-associated virus (AAV), herpes virus, and poxvirus, among others. Viral vectors may be constructed using standard recombinant techniques widely available to one skilled in the art. See, e.g., Molecular cloning: a laboratory manual (Sambrook & Russell: 2000, Cold Spring Harbor Laboratory Press; ISBN: 0879695773), and: Current protocols in molecular biology (Ausubel et al., 1988+ updates, Greene Publishing Assoc., New York; ISBN: 0471625949).

In the fourth aspect, the invention comprises a host cell comprising the nucleic acid according to the second aspect of the invention. Host cells according to the invention may be a cell of bacterial origin, e.g. from Escherichia coli, Bacillus subtilis, Lactobacillus sp., or Caulobacter crescentus, or the aquatic bacteria Yersinia ruckeri, and Vibrio anguillarum, all in combination with the use of bacteria-derived plasmids or bacteriophages for expressing the sequence encoding the polypeptide or protein (as outlined below) according to the invention. The host cell may also be of eukaryotic origin, e.g. yeast-cells (e.g. Saccharomyces or Pichia) in combination with yeast-specific vector molecules; insect cells in combination with recombinant baculo-viral vectors e.g. Sf9 and pVL1393 (Luckow et al., 1988, Bio-technology, vol. 6, p. 47-55); plant cells, in combination with e.g. Ti-plasmid based vectors or plant viral vectors (Barton, et al., 1983, Cell, vol. 32, p. 1033-1043); or mammalian cells also with appropriate vectors or recombinant viruses, such as Hela cells, CHO, CRFK, or BHK cells, or fish cells such as Chinook salmon embryo (CHSE-214) cells, Atlantic salmon cell lines and Rainbow trout cell lines.

Expression of the amino acid sequences according to the first aspect of the invention may also be performed in so-called cell-free expression systems. Such systems comprise all essential factors for expression of the nucleic acid according to the second aspect of the invention, operably linked to a promoter that is capable of expression in that particular system. Examples are the E. coli lysate system (Roche, Basel, Switzerland), or the rabbit reticulocyte lysate system (Promega corp., Madison, USA).

In the fifth aspect, the invention provides an alphavirus comprising the amino acid sequence according to the first aspect of the invention. The alphavirus may be SAV-1, SAV-2, SAV-3, SAV-4, SAV-5, or SAV-6. In one embodiment, the strain is F93-125 (see SEQ ID NO: 4) comprising the E2 protein as described above. The virus according to the invention is attenuated, safe, and elicits immune response that cross-protects against a virulent alphavirus of the same type (e.g., vaccination with the attenuated SAV-3 according to the invention protects against a challenge with a virulent strain of SAV-3). In certain embodiment, the virus elicits cross-protection against SAV of a different type, e.g., the SAV-3 virus according to the invention cross-protects against one or more of SAV-1, SAV-2, SAV-4, SAV-5, or SAV-6.

The viruses according to the invention may be made by introducing a vector comprising the genome of the virus into host cells. The viruses may be grown in tissue culture according to methods known in the art.

In the sixth aspect of the invention, the disclosure provides a vaccine comprising the virus according to any of the embodiments of the fifth aspect of the invention. The virus in the vaccine is preferably live attenuated but it can also be killed. Different methods of virus inactivation are well known in the art and include, without limitation, formalin, binary ethylenimine (BEI) and beta-propiolactone.

If the vaccine comprises a killed virus, it may optionally be adjuvanted. Suitable adjuvants include, without limitation oil emulsions, alum, CpG, saponins, and combinations thereof. If the vaccine comprises a live attenuated virus, it may be non-adjuvanted.

The vaccines of the invention may be monovalent or multivalent. Multivalent vaccines include, in addition to the virus according to the invention, other antigens. Suitable additional antigens include, without limitations, Infectious pancreas necrosis virus (IPNV) Sp, PRV (Piscine orthoreovirus), Piscine myocarditis virus (PMCV), Renibacterium salmoninarum, Tenacibaculum, Infectious salmon anemia virus, Flavobacterium and combinations thereof.

In some embodiments, the vaccine comprises an amount of antigen corresponding to a TCID₅₀ of 10² to 10¹⁰ per dose, or 10³ to 10¹⁰ per dose, preferably a TCID₅₀ of 10⁴ to 10⁹ per dose, more preferably a TCID₅₀ of 10⁵ to 10⁷ per dose.

The vaccine may be in the form of a suspension of the virus or it may be lyophilized. In a lyophilized vaccine it may be useful to add one or more stabilizers. Suitable stabilizers are for example carbohydrates such as sorbitol, mannitol, starch, sucrose, dextran; protein containing agents such as bovine serum or skimmed milk; and buffers such as alkali metal phosphates.

The vaccine of the invention may further comprise a suitable pharmaceutical carrier and/or diluent. Examples of pharmaceutically acceptable carriers or diluents useful in the present invention include sterile water, saline, aqueous buffers such as PBS and others, culture medium, carbohydrates (such as sorbitol, mannitol, starch, sucrose, glucose, dextran), proteins (such as albumin or casein), protein containing agents such as bovine serum or skimmed milk, and buffers (such as phosphate buffer).

In the seventh aspect, the invention provides a method of using the vaccine to elicit a protective immune response against an alphavirus. In certain embodiments, the alphavirus is SAV-2, SAV-3, or SDV. The method comprises administering the vaccine to the fish in need thereof. The species of fish are selected depending on the susceptibility of the fish to the virus. In certain embodiments, the fish are of species Salmo salar (Atlantic salmon) or Oncorhynchus mykiss (trout).

The vaccines according to the invention may be administered to the fish in a variety of ways including parenteral administration, by dipping, immersion or via feed.

It is preferable to administer the vaccine to younger fish, and selected embodiments, the weight of the fish to be vaccinated is at least 5 grams.

In the eighth aspect of the invention, provided is a method of determining whether a sample contains a nucleic acid sequence encoding the amino acid sequence according to any of the embodiments of the first aspect of the invention, the method comprising contacting said sample with a primer or a probe, wherein
a) said primer or probe differentially hybridizes to the nucleic acid sequence encoding at least a portion of SEQ ID NO: 1 depending on whether an amino acid at position 90 of SEQ ID NO: 1 is asparagine encoded by codon aac; and/or
b) said primer or probe differentially hybridizes to the nucleic acid sequence encoding at least a portion of SEQ ID NO: 1 depending on whether an amino acid at position 233 of SEQ ID NO: 1 is threonine; and/or
c) said primer or probe differentially hybridizes to the nucleic acid sequence encoding at least a portion of SEQ ID NO: 1 depending on whether an amino acid at position 375 of SEQ ID NO: 1 is threonine encoded by codon acc.

In certain embodiments, at least 70 percent of the nucleotide in said primer or probe are complementary to the corresponding nucleotides in the template. The complementarity percentage may be greater, e.g., 75%, 80%, 85%, 90%, 95%, or 100%. In certain embodiments, 7, 6, 5, 4, 3, 2, 1, or no nucleotides within the primer or the probe may be non-complementary to the template. The position of the non-complementary nucleotides is not crucial. The non-complementary nucleotide(s) may be at the 3′ of the primer or probe, in the middle of the primer or probe, or at the 5′ end of the primer or probe, or can be interspersed within the primer or probe.

In the embodiments where the method entails the use of the primer which differentially hybridizes to the probe, the mismatches may be at the 3′ end of the primer, as to influence different efficiency of amplification reaction. Thus, if the primer differentially hybridizes to SEQ ID NO: 1 depending on whether the amino acid at position 90 is asparagine encoded by codon aac, then the primer may be designed in such a way that said codon differentially hybridizes to the 3′ portion of the primer. If the primer differentially hybridizes to SEQ ID NO: 1 depending on whether the amino acid at position 233 is threonine, then the primer may be designed in such a way that said codon differentially hybridizes to the 3′ portion of the primer. If the primer differentially hybridizes to SEQ ID NO: 1 depending on whether the amino acid at position 375 is threonine encoded by codon acc, then the primer may be designed in such a way that said codon differentially hybridizes to the 3′ portion of the primer.

In particularly preferred embodiments, the primer or the probe differentially hybridizes to the nucleic acid sequence encoding the amino acid sequence of the invention, wherein in said amino acid sequence of the invention the amino acid at position 90 is aspartic acid, the amino acid at position 233 is absent and/or the amino acid at position is isoleucine.

The primers or the probes according to the invention are generally between 9 and 50 nucleotides long, preferably shorter than 30 nucleotides (e.g., 9-25 nucleotides, 9-20 nucleotides, or 9-15 nucleotides).

In certain embodiments, the primers do not hybridize to the region of the template that includes portions encoding amino acids 90, 233, or 375. The primers may be designed such that amplicons include the areas of the nucleic acid sequence encoding any one of amino acids at position 90, 233, or 375. The length of the amplicon may me generally be 50 bases or longer, e.g., 75 bases or longer, 100 bases or longer, 150 bases or longer, 200 bases or longer, 250 baser or longer, 300 bases or longer, etc.

Once there is sufficient amount of the amplicon, it may contacted with the probe that differentially hybridizes to the nucleic acid sequence encoding the amino acid sequence according to any embodiment of the invention, depending on the nature of the amino acid at position 90 or 375 and/or the nature or the presence of the amino acid at position 233.

Alternatively, if one aims to determine whether the amino acid at position 233 is present or absent, one may analyze the length of the amplicon.

Suitable primers or probes according to the invention may be selected from the group consisting of:

### PCR Primers for Detection of E2 90 Substitution:

| | E2 Fwd a: |
|---|---|
| (SEQ ID NO: 5) | |
| | TATTGCCCCGACTGTGATGGAA |
| | SAV_R15: |
| (SEQ ID NO: 6) | |
| | AGGATGTAGTGGCCGGTGG |

Amplicon size: 230 bp

### PCR—Primers for Detection of E2 233Del:

| | E2 seq Fwd: |
|---|---|
| (SEQ ID NO: 7) | |
| | AACGACAATCAGTGCACAGT |
| | SAV_R16: |
| (SEQ ID NO: 8) | |
| | GGCATTGCTGTGGAAACC |

Amplicon size: 340 bp

### PCR—Primers for Detection of E2 375 Substitution:

| | E2_375_1F: |
|---|---|
| (SEQ ID NO: 9) | |
| | GGTTTCCACAGCAATGCC |
| | E2_375_1R: |
| (SEQ ID NO: 10) | |
| | CGACCAAAGAGTACCGCAC |

Amplicon size: 295 bp

In certain embodiments, the former primer-probe combinations may be used for the determination of the codon encoding an amino acid at position 90, for the deletion of a codon encoding an amino acid at position 233 of SEQ ID NO: 1, and for the determination of the codon encoding an amino acid at position 375, respectively:

| | E2-90 |
|---|---|
| | Forward |
| SEQ ID NO: 11 | |
| | GCGATACCTGGGAAGGGA (Sense) |
| | Probe |
| SEQ ID NO: 12 | |
| | ACAACACGCGGCTTGTGGTG (Sense) |
| | Reverse |
| SEQ ID NO: 13 | |
| | TGCAGCACATCGCACTTT (AntiSense) |
| | E2-Del |
| | Forward |
| SEQ ID NO: 14 | |
| | CCAAGCGACCGTTACCTTTA (Sense) |
| | Probe |
| SEQ ID NO: 15 | |
| | CAGTTTACGTGTGAGGAGCCGGTT (Sense) |
| | Reverse |
| SEQ ID NO: 16 | |
| | ATGATCTAAGGTGCGGCTTG (AntiSense) |
| | E2-375 |
| | Forward |
| SEQ ID NO: 17 | |
| | CGTACCCTTGGGAACTTCTG (Sense) |
| | Probe |
| SEQ ID NO: 18 | |
| | CACACCAAGCACCATCCGGAGTAC (Sense) |
| | Reverse |
| SEQ ID NO: 19 | |
| | AGCAGACCACATGCAACT (AntiSense) |

In certain embodiments, the sample to be used in the method according to this aspect of the invention may be obtained from the fish, such as, for example, Atlantic salmon (Salmo salar). The samples may include, without limitations, blood, muscle tissue, intestinal fluids, heart or kidney tissue, swabs from mucous or gill surfaces.

In other embodiments, the method of the invention can be produced as a quality control to make sure whether the virus according to the invention still retains the mutations at positions 90, 233, and 375 of the E2 protein, and thus is still attenuated.

The following examples are presented as illustrative embodiments but should not be taken as limiting the scope of the invention. Many changes, variations, modifications, and other uses and applications of this invention will be apparent to those skilled in the art.

### Example 1. Isolation of Attenuated Virus

Several clones of wild type SAV 3 isolate, AL V409 (Norway, 2007) attenuated by cell culture passaging have been isolated and sequenced. The differences between the clones and the wild-type virus are provided in in Table 3.

TABLE 3
Overview of amino acid changes in the E2 protein of attenuated
AL V409 clones. The AL V409 wt isolate is used as a reference.

| | E2: | E2: | E2: | E2: | E2: | E2: | E2: | E2: | E2: | E2: | E2: |
|---|---|---|---|---|---|---|---|---|---|---|---|
| Mutation/Isolate | 9 | 90 | 173 | 174 | 175 | 223 | 232 | 233 | 375 | 396 | 437 |
| ALV409 wt | V | N | S | A | P | A | Q | T | T | A | R |
| AL V409 clone | V | D | S | A | P | A | Q | del | I | A | R |
| 1-1-1 | | | | | | | | | | | |
| AL V409 clone | A | N | S | A | P | A | del | T | T | T | R |
| 1-1-2 | | | | | | | | | | | |
| AL V409 clone | V | N | S | A | P | A | Q | T | T | A | R |
| 1-2-1 | | | | | | | | | | | |
| AL V409 clone | V | N | S | A | P | A | Q | T | T | A | C |
| 1-3-2 | | | | | | | | | | | |
| AL V409 | V | N | S | A | P | T | Q | T | T | T | R |
| clone1-4-1 | | | | | | | | | | | |
| AL V1294 | V | D | S | A | P | A | Q | del | I | A | R |
| AL V1296 | V | D | del | del | del | A | Q | del | I | A | R |

Out of these clones, five were selected for further studies: AL V409 clone 1-1-1, AL V409 clone 1-1-2, AL V409 clone 1-2-1, AL V409 clone 1-3-2, and AL V409 clone 1-4-1. Clones AL V1294 and AL V1296 are progenies of ALV 409 1-1-1.

Further comparisons of genomes of these proteins (outside of E2 protein) revealed the following differences:

TABLE 4

| | nsP2: | nsP2 | nsP2: | nsP3: | nsP3: | nsP3: | Capsid: | E3: | E1: |
|---|---|---|---|---|---|---|---|---|---|
| Mutation/Isolate | 246 | 758 | 805 | 31 | 188 | 259 | 269 | 28 | 441 |
| ALV409 wt | A | V | S | D | S | V | K | S | L |
| AL V409 clone | A | V | S | D | S | V | K | S | L |
| 1-1-1 | | | | | | | | | |
| AL V409 clone | A | V | S | D | S | V | K | S | L |
| 1-1-2 | | | | | | | | | |
| AL V409 clone | A | V | S | D | S | V | R | S | L |
| 1-2-1 | | | | | | | | | |
| AL V409 clone | V | V | S | D | P | V | K | P | F |
| 1-3-2 | | | | | | | | | |
| AL V409 | A | V | F | A | S | V | K | S | L |
| clone1-4-1 | | | | | | | | | |
| AL V409 1-1-1 | A | M/A | S | D | S | V | K | S | L |
| AL V1294 | A | A | S | D | S | V | K | S | L |
| AL V1296 | A | A | S | D | S | V | K | S | L |

### Example 2. Screening of the Virulence of Potential Attenuated Candidates

Five candidate attenuated viruses were compared to the parent wild-type AL V409 SAV3 isolate with regards to their ability to induced Pancreas Disease in Atlantic salmon post intraperitoneal injection of 1×10⁵ TCID₅₀/fish. The study was conducted in 12° C. fresh water. Atlantic salmon with an average weight of 34.6 grams were challenged by intraperitoneal injection of each candidate attenuated virus, and monitored for a period of six weeks. The groups injected with different candidate attenuated viruses or parent wild-type were kept in separate tanks throughout the study period. Mortality was registered daily. Heart samples were obtained on RNAlater for 15 fish per group at 2, 4 and 6 weeks post vaccination. These fish were also weighed at the time of sampling. The heart samples on RNAlater were analyzed by PCR to quantify the amount of virus detectable in the heart of the fish at each time point. Also, heart and pancreas samples were obtained from the same fish at the end of the study period, to quantify any tissue damage to the heart and pancreas caused by injection of the candidate attenuated viruses.

Mortality: A single mortality was observed at 27 days post challenge for the group injected with the parent wild-type AL V409 isolate, showing that the parent SAV3 isolate is of low virulence. No fish injected with any of the candidate attenuated viruses died, except for a single mortality being observed in the group injected with candidate AL V409 clone 1-2-1. RT-qPCR identified high SPDV cardiac viral burden (Ct values 17.2 and 18.3) from these fish, verifying that the cause of death for these fish were attributable to the material they were injected with. The remaining four candidates were apparently safe with regards to inability to induce lethal disease in injected Atlantic salmon

Weight: The growth of the groups injected with the various candidate attenuated viruses compared to the parent wild-type isolate is presented in the table below. Two candidates (1-2-1 and 1-3-2) had similar or only mildly improved growth compared to the parent wild-type isolate. One group showed a moderate (and significant) increase in growth (1-4-1) compared to the parent wild-type isolate. Two groups showed a strong (and significant) increase in growth (1-1-1 and 1-1-2) compared to the parent wild-type isolate, where 1-1-1 performed the best. See Table 5.

TABLE 5
Mean weights (in grams, with % growth since vaccination in
parenthesis)) of the groups injected with various candidate
attenuated viruses, compared to the parent wild-type

| Days post | AL V409 | AL V409 | AL V409 | AL V409 | AL V409 | AL V409 |
|---|---|---|---|---|---|---|
| injection | 1-1-1 | 1-1-2 | 1-2-1 | 1-3-2 | 1-4-1 | wild-type |
| 14 | 39.1 | 41.0 | 37.6 | 36.5 | 38.6 | 36.0 |
| | (13.1%) | (18.7%) | (8.8%) | (5.6%) | (11.6%) | (4.1%) |
| 28 | 47.9 | 45.7 | 40.2 | 38.8 | 44.6 | 37.4 |
| | (38.5%) | (32.3%) | (16.3%) | (12.2%) | (29.0%) | (8.3%) |
| 41 | 57.7 | 56.0 | 40.9 | 43.5 | 49.5 | 39.5 |
| | (66.8%) | (62.0%) | (18.3%) | (25.8%) | (43.3%) | (14.2%) |
| p-value vs | <0.0001 | <0.0001 | 0.5978 | 0.0881 | <0.0001 | n.a. |
| wild-type | | | | | | |
| at end | | | | | | |

Cardiac SPDV load: Investigation of cardiac SPDV load at 2, 4 and 6 weeks post injection with the various candidate attenuated viruses detected very large differences compared to the group injected with the parent wild-type isolate, and also between different candidate attenuated viruses as generally described in Hodneland K, Endresen C. J Virol Methods. 2006 February; 131(2):184-92. The results are presented in Table 6 below.

TABLE 6
Prevalence and geometric mean cardiac viral load of fish challenged with various SAV3
AL V409-based potential live attenuated vaccine candidates compared to the parent
wild-type, sampled at 14, 28 and 41 days post challenge (dpc). Statistical analysis
of cardiac viral load for the potential live attenuated vaccine candidates compared
to wild-type was performed by Mann-Whitney test using GraphPad Prism v.8.1.1

| | | AL V409 | AL V409 | AL V409 | AL V409 | AL V409 | AL V409 |
|---|---|---|---|---|---|---|---|
| | | 1-1-1 | 1-1-2 | 1-2-1 | 1-3-2 | 1-4-1 | wild-type |
| 14 dpc | Prevalence | 33% | 93% | 93% | 100% | 100% | 100% |
| | Geometric | 33.1 | 22.2 | 17.2 | 17.0 | 21.8 | 15.9 |
| | mean Ct | | | | | | |
| | p-value | <0.0001 | <0.0001 | 0.3189 | 0.0656 | <0.0001 | — |
| | against wt | | | | | | |
| 28 dpc | Prevalence | 0% | 93% | 100% | 100% | 93% | 100% |
| | Geometric | 34.6 | 25.7 | 23.8 | 24.6 | 27.5 | 22.1 |
| | mean Ct | | | | | | |
| | p-value | <0.0001 | 0.0003 | 0.0627 | 0.0009 | <0.0001 | — |
| | against wt | | | | | | |
| 41 dpc | Prevalence | 20% | 80% | 100% | 93% | 93% | 100% |
| | Geometric | 33.6 | 29.8 | 25.8 | 27.0 | 30.7 | 25.4 |
| | mean Ct | | | | | | |
| | p-value | <0.0001 | <0.0001 | 0.5940 | 0.1362 | <0.0001 | — |
| | against wt | | | | | | |

Two candidates showed cardiac viral levels comparable to or only moderately lower than the parent wild-type, demonstrating no degree of attenuation with practical relevance (clones 1-2-1 and 1-3-2). Two candidates demonstrated significantly lower cardiac viral burden compared to the parent wild-type (1-1-2 and 1-4-1), but the majority of the fish in the groups injected with these candidates were still positive for virus at termination of the study 6 weeks post injection. The candidate showing the highest degree of attenuation was AL V409 1-1-1, showing a significant reduction in cardiac viral load compared to the parent wild-type isolate throughout the study. At the first sampling point 14 days post challenge, it was not possible to detect the virus in the majority of the fish (67%) injected with this candidate. At the second sampling point 14 days post injection, the candidate was undetectable in the heart of all fish sampled. At the final sampling point 41 days post injection, the virus was detectable in the heart of 20% of the fish injected with this candidate. Molecular analyses revealed that it was the wild-type virus that was detected in these fish 41 days post injection with candidate attenuated virus AL V409 1-1-1, and not the candidate attenuated virus. This was likely caused by the candidate attenuated virus still containing a low-level impurity of wild-type strain, as it had not been subject to purification to remove all traces of wild-type prior to start of the study. The candidate attenuated virus was not detectable at the time of termination of the study 6 weeks post injection.

Histopathology: The severity of tissue damage developing to the heart and pancreas post injection with the various candidate attenuated viruses and the wild-type strain was investigated by histopathological examination on samples collected at termination of the study. This was performed by a third-part assessor (PHARMAQ Analytiq) without knowledge of the study design or purpose of the study. The results are presented in the table 7 below. The results were quite consistent with the results for the other parameters investigated in the trial. Three of the candidates induced tissue damage to the heart and pancreas to a comparable extent (1-2-1 and 1-3-2) or only moderately reduced (1-4-1) compared to the parent wild-type isolate. Candidate 1-1-2 showed a strong reduction in tissue damage to the pancreas compared to the parent wild-type isolate, but still had some residual virulence, with some pathology being observed, also for the heart. No tissue damage to the pancreas was detected for fish injected with candidate AL V409 1-1-1, and only mild unspecific findings were detected in the heart for fish injected with this candidate.

TABLE 7
Mean histopathology score on a scale of 0-3 (with minimum and maximum
observed values in parenthesis) 6 weeks post injection with various candidate
attenuated viruses, compared to the parent wild-type AL V409.

| | AL V409 | AL V409 | AL V409 | AL V409 | AL V409 | AL V409 |
|---|---|---|---|---|---|---|
| Tissue | 1-1-1 | 1-1-2 | 1-2-1 | 1-3-2 | 1-4-1 | wild-type |
| Pancreas | 0.00 | 0.43 | 2.68 | 2.90 | 1.93 | 2.89 |
| | (0.0-0.0) | (0.0-2.0) | (0.0-3.0) | (2.0-3.0) | (0.0-3.0) | (2.0-3.0) |
| Heart | 0.07 | 0.20 | 0.32 | 0.20 | 0.07 | 0.07 |
| compactum | (0.0-0.5) | (0.0-1.0) | (0.0-1.5) | (0.0-1.0) | (0.0-0.5) | (0.0-1.0) |
| Heart | 0.03 | 0.27 | 0.43 | 0.30 | 0.07 | 0.86 |
| spongiosa | (0.0-0.5) | (0.0-1.0) | (0.0-1.5) | (0.0-1.0) | (0.0-1.0) | (0.0-2.5) |
| Heart | 0.33 | 0.79 | 0.50 | 0.50 | 0.63 | 0.23 |
| atrium | (0.0-1.0) | (0.0-2.0) | (0.0-2.0) | (0.0-2.0) | (0.0-1.5) | (0.0-1.0) |

Conclusions: The study identified a promising live attenuated vaccine candidate of SAV3 (AL V409 1-1-1) with a highly attenuated phenotype compared to the parent wild-type strain. From the data above, it appears that three mutations in E2 gene (at positions 90, 233, and 375) are sufficient to confer the attenuated phenotype.

### Example 2: Safety of Screening of Purified Vaccine Candidate

The candidate AL V409 1-1-1 was subject to extensive purification to remove any trace of wild-type contamination, and a study investigating the safety of the purified vaccine candidate (AL V409 1-1-1-2) was performed. Atlantic salmon weighing 23.1 grams in average were vaccinated by intraperitoneal injections of the candidate in a dose of 3.8×10⁶ TCID₅₀/fish. The study was conducted in 12° C. freshwater. The fish were monitored for a period of 6 weeks post vaccination. 10 fish were sampled at each of time points 5, 8, 11, 14, 21, 28, 35 and 42 days post vaccination, except for the final sampling point when only 9 fish were available for sampling. The fish were weighed, and heart, pancreas and kidney samples were obtained on RNAlater for quantification of viral burden. Also, heart and pancreas samples were obtained on formalin from the same fish at all time points to quantify any tissue damage developing post vaccination.

Mortality: there was no mortality observed throughout the 42 days observation period post vaccination.

Weight: The fish used for sampling at each time point were weighed, and the results are presented in table 8 below (grams, and percent growth since vaccination). No negative control group was included in the study which would have enabled to accurately quantify any potential negative effect on growth. Despite this, the growth of the fish injected with the live attenuated vaccine candidate was as expected from healthy fish, showing no apparent residual virulence with regards to growth post vaccination.

TABLE 8
Growth post vaccination with live attenuated vaccine candidate AL V409 1-1-1-2

| Days post | | | | | | | | |
|---|---|---|---|---|---|---|---|---|
| injection | 5 | 8 | 11 | 14 | 21 | 28 | 35 | 42 |
| Mean | 22.6 | 23.7 | 25.0 | 25.4 | 29.7 | 31.8 | 33.5 | 36.6 |
| weight | | | | | | | | |
| (gram) | | | | | | | | |
| Percent | −2.4% | 2.4% | 8.3% | 10.0% | 28.5% | 37.4% | 45.0% | 58.5% |
| growth | | | | | | | | |

Viral load to the heart, pancreas and kidney: SPDV viral load to the heart, pancreas and kidney was investigated by RT-qPCR to investigate safety and tissue distribution of the vaccine candidate post vaccination. The results are presented in Table 9 below. The live attenuated vaccine candidate was detectable in the fish in the target organs (heart and pancreas) up to 14 days post vaccination, after which it was no longer detectable. In kidney, the live attenuated vaccine candidate was undetectable after 8 days post vaccination.

TABLE 9
SPDV viral load to the heart, pancreas and kidney post vaccination,
presented as prevalence at each time point and tissue

| Days post | | | | | | | | |
|---|---|---|---|---|---|---|---|---|
| injection | 5 | 8 | 11 | 14 | 21 | 28 | 35 | 42 |
| Heart | 40% | 10% | 40% | 20% | 0% | 0% | 0% | 0% |
| Pancreas | 30% | 70% | 30% | 10% | 0% | 0% | 0% | 0% |
| Kidney | 40% | 30% | 0% | 0% | 0% | 0% | 0% | 0% |

Histopathology: No specific findings associated with Pancreas Disease was detected at any time point by histopathological examination, showing that the vaccine was safe for the vaccinated fish. An inflammatory response was observed in the proximity of blood vessels and pancreatic tissue in the pyloric area in the samples obtained at 11 days post vaccination, in 5/10 investigated fish. The pancreas tissue was not affected, and this did not cause lasting damage, as no pathology was observed in pancreas (or heart) at later sampling points.

Conclusions: The live attenuated vaccine candidate was safe to use for intraperitoneal vaccination of Atlantic salmon in 12° C. fresh water in a dose of 3.8×10⁶ TCID₅₀/fish.

### Example 3: Dose-Response of Live Attenuated Vaccine Candidate AL V409 1-1-1 Against Cohabitation Challenge with SAV2 and SAV3 in Sea Water

A study was conducted to investigate the efficacy of live attenuated vaccine candidate AL V409 1-1-1 against cohabitation challenge with SAV2 and SAV3 in sea water. Atlantic salmon with an average weight of 23.9 grams were vaccinated by intraperitoneal injections of AL V409 1-1-1 in doses of 6.7×10⁴, 6.7×10⁵ or 6.7×10⁶TCID₅₀/fish. Three other groups were included in the study: commercial monovalent inactivated oil-adjuvanted PD vaccine (ALPHA JECT® micro 1 PD, PHARMAQ part of Zoetis), negative control substance (Phosphate buffered Saline) and uninjected fish to be used as shedder fish during cohabitation challenge. Immunization was performed in 12° C. fresh water. Starting from one week post vaccination, the fish were smoltified by exposure to continuous light for a 6 weeks period, after which they were challenged in sea water. The fish vaccinated with the live attenuated vaccine candidate were kept in a different tank during the immunization period than the fish vaccinated with the negative and positive control substance, and the shedder fish. The fish were allocated to two identical tanks at the time of challenge, each containing 25 fish per group. The groups were challenged by cohabitation challenge, where naïve fish were intraperitoneally injected with either a SAV2 isolate (AL V1237) or a SAV3 isolate (AL V413) and added to each tank. The SAV2 and SAV3 challenge was performed in different tanks. Efficacy was measured by investigation of mortality, growth, cardiac viral load (by RT-qPCR) and tissue damage to the heart and pancreas (by histopathological examination) at termination of the study 4 weeks post challenge.

Mortality: no mortality was observed during the challenge period for neither vaccinated nor control fish. The only group experiencing mortalities were the intraperitoneally injected shedder fish, which is expected.

Weight: The growth of the groups injected with 6.7×10⁴-6.7×10⁶ TCID₅₀/dose of live attenuated vaccine candidate AL V409 1-1-1 was compared to the growth of fish vaccinated with the negative and positive control substances. The results are described in Table 10 below. All fish were weighed at the start and termination of the challenge to accurately quantify growth during the challenge period. The results show that all groups vaccinated with 6.7×10⁴-6.7×10⁶ TCID₅₀/dose of ALV409 1-1-1 and the positive control substance showed improved growth during the observation period compared to the negative control substance, demonstrating that the live attenuated vaccine candidate protects against PD-related reduction in weight gain during a PD challenge.

TABLE 10
Median weights (in grams) at the time of challenge and termination of challenge
4 weeks later, and median percent growth during the observation period.

| | | AL V409 1-1-1 | AL V409 1-1-1 | AL V409 1-1-1 | | |
|---|---|---|---|---|---|---|
| | | 6.7 × 10⁶ | 6.7 × 10⁵ | 6.7 × 10⁴ | AJm1PD | PBS |
| SAV2 | Median weight | 53.0 | 57.9 | 55.9 | 58.2 | 56.3 |
| | at challenge | | | | | |
| | Median weight | 76.8 | 79.5 | 78.0 | 80.0 | 73.1 |
| | at termination | | | | | |
| | % growth | 44.9% | 37.2% | 39.5% | 37.5% | 29.8% |
| | p-value against | 0.3423 | 0.0003 | 0.1775 | 0.1023 | n.a. |
| | PBS termination | | | | | |
| SAV3 | Median weight | 56.2 | 55.1 | 55.7 | 58.1 | 58.4 |
| | at challenge | | | | | |
| | Median weight | 81.3 | 81.8 | 83.0 | 87.7 | 71.0 |
| | at termination | | | | | |
| | % growth | 44.7% | 48.5% | 49.0% | 50.9% | 21.6% |
| | p-value against | <0.0001 | <0.0001 | <0.0001 | <0.0001 | n.a. |
| | PBS termination | | | | | |

Prevalence of SPDV in fish at termination of challenges after a 4 weeks observation period: The SPDV cardiac viral load for fish experiencing a 4 weeks cohabitation challenge with SAV2 or SAV3 in sea water was investigated for all surviving fish at termination of the study (n=25 per group per challenge strain). The geometric mean Ct value and the prevalence per group was calculated. The results are presented in Table 11 below. Fish vaccinated with even the lowest dose investigated of AL V409 1-1-1 (6.7×10⁴ TCID₅₀/fish) showed a highly efficient reduction in SPDV cardiacviral load compared to the PBS control group against both SAV2 and SAV3 challenge, which was statistically significant (p<0.0001). Also the positive control group provided efficient protection against SAV2 and SAV3 infection.

TABLE 11
Prevalence of SPDV in heart tissue at termination
of the challenges 4 weeks post challenge.

| | | AL V409 1-1-1 | AL V409 1-1-1 | AL V409 1-1-1 | | |
|---|---|---|---|---|---|---|
| | | 6.7 × 10⁶ | 6.7 × 10⁵ | 6.7 × 10⁴ | AJm1PD | PBS |
| SAV2 | Prevalence | 4.0% | 0.0% | 13.0% | 4.0% | 77% |
| | Geometric | 34.5 | 34.6 | 33.1 | 34.5 | 20.3 |
| | mean Ct | | | | | |
| | p-value | <0.0001 | <0.0001 | <0.0001 | <0.0001 | n.a. |
| | against PBS | | | | | |
| SAV3 | Prevalence | 0.0% | 0.0% | 0.0% | 4.0% | 92.0% |
| | Geometric | 34.6 | 34.6 | 34.6 | 33.6 | 19.1 |
| | mean Ct | | | | | |
| | p-value | <0.0001 | <0.0001 | <0.0001 | <0.0001 | n.a. |
| | against PBS | | | | | |

Histopathology: The severity of tissue damage to the heart and pancreas was investigated by histopathological examination for all surviving fish at termination 4 weeks post challenge. The results are presented in table 12 below. The results were highly consistent with the weight and PCR data. For both the SAV2 and SAV3 challenges, all vaccinated fish were significantly protected against development of cardiac tissue damage and pancreatic damage compared to the PBS control fish (p<0.0001), with near perfect protection observed for all doses tested of AL V409 1-1-1, and the positive control vaccine. No obvious differences between the vaccines were observed.

TABLE 12
Mean histopathology score on a scale of 0-3 (with minimum and maximum observed values
in parenthesis) at termination 4 weeks post onset of cohabitation challenge. Statistical
comparison between groups by Mann-Whitney test (GraphPad Prism v.8.1.1)

| | | AL V409 1-1-1 | AL V409 1-1-1 | AL V409 1-1-1 | | |
|---|---|---|---|---|---|---|
| | | 6.7 × 10⁶ | 6.7 × 10⁵ | 6.7 × 10⁴ | AJm1PD | PBS |
| SAV2 | Mean | 0.00 | 0.04 | 0.00 | 0.00 | 1.96 |
| | Pancreas | (0.0-0.0) | (0.0-1.0) | (0.0-0.0) | (0.0-0.0) | (0.0-3.0) |
| | p-value | <0.0001 | <0.0001 | <0.0001 | <0.0001 | n.a. |
| | against PBS | | | | | |
| | Mean heart | 0.00 | 0.00 | 0.00 | 0.00 | 1.10 |
| | compactum | (0.0-0.0) | (0.0-0.0) | (0.0-0.0) | (0.0-0.0) | (0.0-3.0) |
| | p-value | <0.0001 | <0.0001 | <0.0001 | <0.0001 | n.a. |
| | against PBS | | | | | |
| | Mean heart | 0.04 | 0.00 | 0.02 | 0.00 | 1.23 |
| | spongiosa | (0.0-0.5) | (0.0-0.0) | (0.0-0.5) | (0.0-0.0) | (0.0-3.0) |
| | p-value | <0.0001 | <0.0001 | <0.0001 | <0.0001 | n.a. |
| | against PBS | | | | | |
| | Mean heart | 0.63 | 0.75 | 0.52 | 0.41 | 0.69 |
| | epicardium | (0.0-1.0) | (0.0-1.5) | (0.0-1.0) | (0.0-1.0) | (0.0-2.0) |
| | p-value | 0.6941 | 0.6532 | 0.1952 | 0.0516 | n.a. |
| | against PBS | | | | | |
| SAV3 | Mean | 0.16 | 0.00 | 0.00 | 0.13 | 2.64 |
| | Pancreas | (0.0-2.0) | (0.0-0.0) | (0.0-0.0) | (0.0-3.0) | (0.0-3.0) |
| | p-value | <0.0001 | <0.0001 | <0.0001 | <0.0001 | n.a. |
| | against PBS | | | | | |
| | Mean heart | 0.02 | 0.00 | 0.00 | 0.08 | 1.72 |
| | compactum | (0.0-0.5) | (0.0-0.0) | (0.0-0.0) | (0.0-2.0) | (0.0-3.0) |
| | p-value | <0.0001 | <0.0001 | <0.0001 | <0.0001 | n.a. |
| | against PBS | | | | | |
| | Mean heart | 0.02 | 0.00 | 0.00 | 0.08 | 2.36 |
| | spongiosa | (0.0-0.5) | (0.0-0.0) | (0.0-0.0) | (0.0-2.0) | (0.0-3.0) |
| | p-value | <0.0001 | <0.0001 | <0.0001 | <0.0001 | n.a. |
| | against PBS | | | | | |
| | Mean heart | 0.70 | 0.64 | 0.46 | 0.48 | 1.12 |
| | epicardium | (0.0-1.5) | (0.0-1.0) | (0.0-1.0) | (0.0-2.0) | (0.5-2.0) |
| | p-value | 0.0010 | <0.0001 | <0.0001 | <0.0001 | n.a. |
| | against PBS | | | | | |

Conclusions: The live attenuated vaccine candidate AL V409 1-1-1 induces protective immunity against SAV2 and SAV3 cohabitation challenge in sea water when used in a dose of >6.7×10⁴ TCID₅₀/fish.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

1. An amino acid sequence comprising SEQ ID NO: 1, or a sequence at least 94% identical thereto, with the proviso that an amino acid at position 233 is not threonine or is absent.

2. The amino acid sequence according to claim 1 wherein the amino acid at position 90 is not asparagine.

3. The amino acid sequence according to claim 2 wherein the amino acid at position 90 is aspartic acid or glutamic acid.

4. The amino acid sequence according to claim 3, wherein the amino acid at position 90 is aspartic acid.

5. The amino acid sequence according to claim 1, wherein the amino acid at position 233 is absent.

6. The amino acid sequence according to claim 1, wherein further amino acid at position 375 is not threonine.

7. The amino acid sequence according to claim 6, wherein the amino acid at position 375 is isoleucine or leucine.

8. The amino acid sequence of claim 7, wherein the amino acid at position 375 is isoleucine.

9. The amino acid sequence according to claim 1, wherein:
a) an amino acid at position 173 is serine or a conservative substitute thereof; and/or
b) an amino acid at position 174 is alanine or a conservative substitute thereof; and/or
c) an amino acid at position 175 is proline or a conservative substitute thereof.

10. The amino acid sequence according to claim 9, wherein
a) the amino acid at position 173 is serine or a conservative substitute thereof;
b) the amino acid at position 174 is alanine or a conservative substitute thereof; and
c) the amino acid at position 175 is proline or a conservative substitute thereof

11. The amino acid sequence of claim 1 which is at least 95% or at least 98% or at least 99% identical to SEQ ID NO: 1.

12. The amino acid sequence according to claim 1, comprising SEQ ID NO: 2.

13. The amino acid sequence according to claim 1, wherein:
a) an amino acid at position 173 is absent; and/or
b) an amino acid at position 174 is absent; and/or
c) an amino acid at position 175 is absent.

14. The amino acid sequence according to claim 11, wherein
a) an amino acid at position 173 is absent; and
b) an amino acid at position 174 is absent; and
c) an amino acid at position 175 is absent.

15. A nucleic acid sequence encoding the amino acid sequence of claim 1.

16. The nucleic acid sequence of claim 15, comprising SEQ ID NO: 3.

17. A vector comprising the nucleic acid sequence according to claim 15.

18. A host cell comprising the nucleic acid sequence of claim 15.

19. A salmonid alphavirus comprising the amino acid sequence according to claim 1.

20. A salmonid alphavirus comprising the nucleic acid sequence according to claim 15.

21. The salmonid alphavirus of claim 19 which is Salmonid alphavirus 3.

22. The salmonid alphavirus according to claim 19, comprising strain F93-125.

23. A vaccine comprising an attenuated salmonid alphavirus according to claim 21.

24. The vaccine according to claim 23, which is non-adjuvanted.

25. The vaccine according to claim 23 further comprising a different strain of salmonid alphavirus.

26. The vaccine according to claim 23 further comprising at least one additional antigen selected from the group consisting of Infectious pancreas necrosis virus (IPNV) Sp, PRV (Piscine orthoreovirus), Piscine myocarditis virus (PMCV), Renibacterium salnoninarum, Tenacibaculum, Infectious salmon anemia virus, Flavobacterium and combinations thereof.

27. A method of protecting fish against pancreas disease, comprising administering to a fish in need thereof the composition according to claim 23.

28. (canceled)

29. The method of according to claim 27 or the use according to claim 28,
wherein said fish in need thereof is Atlantic salmon (Salmo salar).

30. A method of determining whether a sample contains a nucleic acid sequence encoding the amino acid sequence according to claim 2, the method comprising contacting said sample with a primer or a probe, wherein
a) said primer or probe differentially hybridizes to the nucleic acid sequence encoding at least a portion of SEQ ID NO: 1 depending on whether an amino acid at position 90 of SEQ ID NO: 1 is asparagine encoded by codon aac;
b) said primer or probe differentially hybridizes to the nucleic acid sequence encoding at least a portion of SEQ ID NO: 1 depending on whether an amino acid at position 233 of SEQ ID NO: 1 is threonine.

31. A method of determining whether a sample contains a nucleic acid sequence encoding the amino acid sequence according to claim 6, the method comprising contacting said sample with a primer or a probe wherein:
a) said primer or probe differentially hybridizes to the nucleic acid sequence encoding at least a portion of SEQ ID NO: 1 depending on whether an amino acid at position 90 of SEQ ID NO: 1 is asparagine encoded by codon aac;
b) said primer or probe differentially hybridizes to the nucleic acid sequence encoding at least a portion of SEQ ID NO: 1 depending on whether an amino acid at position 233 of SEQ ID NO: 1 is threonine; or
c) said primer or probe differentially hybridizes to the nucleic acid sequence encoding at least a portion of SEQ ID NO: 1 depending on whether an amino acid at position 375 of SEQ ID NO: 1 is threonine encoded by codon acc.

32. The method of claim 30, wherein said portion of SEQ ID NO: 1 comprises an amino acid at position 90, and wherein said primer or probe differentially hybridizes to the nucleic acid sequence encoding a portion of SEQ ID NO: 1 depending on whether said amino acid at position 90 is asparagine encoded by codon aac or aspartic acid encoded by codon gac.

33. The method of claim 30, wherein said portion of SEQ ID NO: 1 comprises an amino acid at position 233, and wherein said primer or probe differentially hybridizes to the nucleic acid sequence encoding a portion of SEQ ID NO: 1 depending on whether said amino acid at position 233 is threonine or absent.

34. The method of claim 30 wherein said portion of SEQ ID NO: 1 comprises an amino acid at position 375, and wherein said primer or probe differentially hybridizes to the nucleic acid sequence encoding a portion of SEQ ID NO: 1 depending on whether said amino acid at position 375 is threonine encoded by codon acc or isoleucine encoded by codon atc.

35. The method of claim 30 wherein said primer or probe is 9-40 nucleotides long.

36. A method of determining whether a sample contains a nucleic acid sequence encoding the amino acid sequence according to claim 4, the method comprising contacting said sample with a pair of primers, amplifying a nucleic acid sequence that encodes a portion of SEQ ID NO: 1, said portion comprising an amino acid at position 233; and determining the length of the amplified portion of said nucleic acid sequence.

37. The method of claim 36, wherein the length of the amplified portion of said nucleic acid sequence is 50-250 nucleotides.

38. The method according to claim 30, wherein said sample is a sample obtained from a fish.

39. The method according to claim 38, wherein said fish is Atlantic salmon (Salmo salar).

40. The method according to claim 30 wherein said sample is obtained from a cell culture or a tissue culture.


SEQUENCE LISTING (19 sequences)

SEQ ID NO: 1 - protein, 438 aa, Salmonid Alphavirus 3
AVSASPAAVYDTQILAAHAAASPYRAYCPDCDGTACISPIAIDEVVSSGSDHVLRIRVGSQSGVTAKGGAAGETSLRYLGRDGKVHAADNTRLVVRTTAKCDVLQATGHYILASCPEGQSITVAATLDGTRHQCTTVFEHQVTEKFTRERSKGHHLSDLTKKCTRFSTTPKKSAPYLVDVYDALPISVEISTVVTCNDNQCTVRVSPGTTVKFDKKCKSAAQATVTFTSDSQTFTCEEPVLTAASITQGKPHLRSSMLPSGGKEVKARIPFPFPPETATCRVSVAPLPSITYEESDVLLAGTAKYPVLLTTRNLGFHSNATSEWIQGKYLRRIPVTPQGIELTWGNNAPLHFWSSVRYASGDADAYPWELLVHHTKHHPEYAWAFVGVACGLLVIAVCMFACACNRVRYSLVANTFNPNPPPLTALTAALCCIPGARA

SEQ ID NO: 2 - protein, 437 aa, synthetic construct
  REGION 1..437: note = Mutated SAV-3 protein
AVSASPAAVYDTQILAAHAAASPYRAYCPDCDGTACISPIAIDEVVSSGSDHVLRIRVGSQSGVTAKGGAAGETSLRYLGRDGKVHAADDTRLVVRTTAKCDVLQATGHYILASCPEGQSITVAATLDGTRHQCTTVFEHQVTEKFTRERSKGHHLSDLTKKCTRFSTTPKKSAPYLVDVYDALPISVEISTVVTCNDNQCTVRVSPGTTVKFDKKCKSAAQATVTFTSDSQFTCEEPVLTAASITQGKPHLRSSMLPSGGKEVKARIPFPFPPETATCRVSVAPLPSITYEESDVLLAGTAKYPVLLTTRNLGFHSNATSEWIQGKYLRRIPVTPQGIELTWGNNAPLHFWSSVRYASGDADAYPWELLVHHIKHHPEYAWAFVGVACGLLVIAVCMFACACNRVRYSLVANTFNPNPPPLTALTAALCCIPGARA

SEQ ID NO: 3 - DNA, 1311 bp, synthetic construct
  misc_feature 1..1311: note = Nucleic acid sequence endoding mutated protein of Salmonidalphavirus 3
  source 1..1311: mol_type = other DNA
gctgtgtctgcgtcgcctgccgccgtttacgacacacaaatcctcgccgcccacgcagctgcctccccgtacagggcgtattgccccgactgtgatggaactgcctgcatctcgccgatagctattgacgaggtggtaagcagcggtagcgaccacgtcctccgcatccgggtcggttctcaatcgggagtgaccgctaaaggcggtgcggcgggtgaaacatctctgcgatacctgggaagggacggtaaggtgcacgccgcggacgacacgcggcttgtggtgcgcaccactgcaaagtgcgatgtgctgcaggccaccggccactacatcctggccagctgcccagaggggcagagtattactgttgcggccacactggacggcacccgccaccaatgcaccacggttttcgaacatcaagtaacggagaagttcaccagagaacgcagcaagggccaccacttgtccgacctgaccaagaagtgcaccagattttccaccaccccgaagaagtccgccccctacctcgttgacgtgtacgacgctctgccgatttctgtagagattagcaccgttgtaacatgcaacgacaatcagtgcacagtgagggtgtcacccggtaccacagtgaaattcgataagaagtgcaagagcgctgcccaagcgaccgttacctttaccagcgactcccagtttacgtgtgaggagccggttctgacggccgccagtatcacccagggcaagccgcaccttagatcatctatgttgcccagcggaggcaaggaagtgaaggcgaggatcccattcccgttcccgccagagaccgcgacctgcagagtaagtgtcgccccgctgccgtcgatcacctatgaggaaagcgacgttctgctggccggtaccgcgaagtaccccgtgctgctgactacacggaaccttggtttccacagcaatgccacatccgaatggatccagggtaagtacttgcgccgtatcccggtcacgccccaagggatcgaactaacgtggggaaataacgcaccgttgcacttctggtcatctgttaggtacgcatctggggacgccgacgcgtacccttgggaacttctggtgcaccacatcaagcaccatccggagtacgcgtgggcgtttgtaggagttgcatgtggtctgctggttattgcagtatgcatgttcgcgtgcgcatgcaacagagtgcggtactctttggtcgccaacacgttcaacccgaacccaccaccactgaccgcactgactgcagcattgtgctgcatacctggggctcgtgcg

SEQ ID NO: 4 - DNA, 11919 bp, Salmonid Alphavirus
  misc_feature 1: note = n is a, t, c, or g
  source 1..11919: mol_type = genomic DNA
nttaaatccagaagcatacatatatcaatgatgcaaaatctcacagctaacccctccgccggcgctacagttactgtaaatctgcccgcggaccacccggccctgaaccagttcaagactgcgtttccagggttcgaagtggtggccagcaataggtcgtctaacgaccatgccgccgccagagctttctcccacttggctaccaagtggattgagcgcgacatcggtggccgtcaggtcatcgttgccgacatagggagcgcaccagcgagaagaatcggggcacccgataacgtcacataccacagtgtgtgtcctcgcaaatgcgctgaagaccctgagaggctggcttcttatgccaggaaactcgtcagggctgtggagagaggagatggacacctcgtcaatgaaaaaatcactgacttgaaggatgttctggagaacccggacacgtcactggaaaccaccagtatctgcctgaacgacgacgtgagttgtaaagtaaaggccgatattgcagtgtaccaggatgtgtacgcagtcgatgctccgtctaccatctacgcgcaggccgacaaaggtacgcgagtggtatactggatcgggttcgagccgttcgtgttccacacggacgctatggcgggtagcttcccgctatatgacgccaactggagcgactcggcggtactggcagctaaaaacctgccactgtgctactcagggttgtcggaggattccattaaatggagattccgtttccgtgacaagccactggtaccgtctggagaaatccactactcggtgggtagcacccactacgttgaggaccgtgacaagctgaaaagctggcacctgccttctaccttccacttcgtggcacccaacaagtatacatgccgttgcgatacggtggtaagctgtggcggctacgtcgtaaagaagatcacgatctgtgagggtatcgtcggtattcccgcgaaggaggaactggccacctcgtaccaccgtgatggagtggtggtgaccaagttctctgatacgatcaaccacgaacaggtgtcattcccagtggtgacctacatccccgccgtcatctgtgatcagatgaccgccatgacggccaatcccgtcaaatactcggatgccgtcaaactgctggtaggactcaaccaaaggatcgtggtcaacgggaccaccgtccgcaacgtcaactccatggataactcgctgattcccgtattcgctcgcgcattatgcagctgggcggacgaagttcggcgggacatggaggacgaacaagacctgtacggtatcacgtccgtcaccacatggatctgtatctgcagagcgtatgacaaacgccagcagcataccttctaccgtaggcccaagcagtcctcgggcatatatgtgcctgccaagttcacagggtcgctgagagccgccctctcagccacctaccttaacctgcctctgaagcaactgctgcttaatacgctcaaacgtgcgattaaacctatggatcaggccatcgcagatgagacagaagcacttgcgcacgacgcagctgaggtgcacgagttgacagaggaggagcgcaggcaacaggccgccaatcctagttacatcgcagacgtgctgggccaagacgacgacgaggaggaagccggcgacggcatgtctgatgtggacctcggcgaagaagacggggcgggtgccaccatcatcgattgtcaaagaggcaccgtgaaagtaatcacggcgttcggtgacaacatgatgggggaatacctagtattgtccccggtgaccgtgttgcgcaccaggaaactggccattctgctcggaccactcgcggaagaagtgatgcagtatgtccacaaaggccggaccgggcgctatgccattgagaagaataacctgaaggtactcatccctacgggggtgtccctcaagaccgatcacttccaggcccttgccgaaagtgctaccttgacctacaacgactacctgttcacatgccgcacgctcgaccagctggcaacaagaggatcagcaagaaacaccgacgaagtctactacaagcttgttgacgccgctaaggcgagggacgaatacgtttacgaactgtcctccaagcaatgcgttaagaaagaagatgcaacaggcacagtcctccaaggggacatatgcaatccgccttatcaccagtttgcctacgaggcgctgcgtaaaaggccggctcacacacacgacgtccacacgattggcatctacggggttccgggtgcaggaaaaaccgctatcatcacaaccgaagtaactacacgcgacttggtcgctagtggaaaaaaggagaattgtgaagacatcaagcggtgcgtgttggagagacgcggcttgaaaatagccgcacgtacggttgactcactgttctacggtgcataccgaggagcggtcaatacgctgtacgttgacgaggcttacgcctgtcactcgggcactctgctcgccttgatcgctgcggtaagaccgacgggaaaggtggtgctttgtggtgaccctaaacaagtcggatgtgtgaaccagctccaaatgcggatgcattacaaccatgagattagtgaccgcgttttgcggaagaacatctcccgccgttgcactcatacgcttacggccatcgtttcaaacctcaactacgaaggtagaatgaagactacgaacccgtgcaaaaaacccgtactaattgacaccaccggatccaccaagcccgacaaggaagcgttggtgttgacgtgcttccgcgggtgggtcaaggatctaaaattcctctatcctcacaacgagctcatgactgcggccgcttcgcagggcctgacccgcgaaaaggtgtatgccgttcgttgtcgtgtcacgacgaacccactctatgagccgacctctgagcacattactgtccttttgacgcgcaccaacgacgaactggtatggaagacactgccaaacgacccgctgatccctatactctccaagccaccgaaaggagactactccgctaccatggaggattgggaggatgaacacaacggtatcctggctgccctcagagaagcgtgtgtcccacggatgaatttcgcgcatgggaagcgtaatacctgttgggcagttacgagcagccgggtgctgcatgaggcgggcgtccagataacgccggaggactataaccgtatctttccggcgtttcgagaggacaagccgcattcggctctggcagccttggatgccgtcgctactctcgtgtggggcttggatacttcctcgggcattctgagtggaaaaggcagcttcatgcggctggagaacagccactggagtaactccaacagggggtacgagtacggcctcaacctggacgcactggaaggctatgagattgccaatccacgcatgattaaagcgcttaaacaacggcgcgggcgcgaatgttacgacatcgaaacggggaagttggtgcccctggatcctgccagggtccaagtgccgattaaccgaatcgtgccccacgtgttggtggacacttctgcagcggccaaaccaggcttcctggaaaacagactcacagttgacagatgggaccaagtgcacagcttcaagacaagagcagcagtcaagtttgcagaactgacgaagcgtgtctcatataactcagtccttgaccttggagccgccccgggaggagtgaccgactactgcgtgaagaaaggaaaaaccgtcacaagcgtctccgagcagtgggatactaagccgagaggcgctgtggtcgttacggcagatatcaacggcccacttaacaacctaggtatcttcgacctagtgttttgtgacgcagctggaccacgacgctatcaccactacgcgcagtgcgaagaccacgccgtgctgttcacatcagcatgcaaacatggggtggagcgcacagccaagggcggagtcttcattgtgaaggcgtacggaatggcggaccgccgaacagagagggcagtggaaggtacagcgaggtatttcaggtccgtttcggtcgaaaagcccgtctcctcccgtataaccaacgtggaagtcttcttcaagttctctggcagatgtcgcccacacgcacgttccatcgcacatttgggccctcaactgaccgacatttacgcccgtacgtggaaggcgtataaaatgctggcgagaggcagtgtcgctgacaaggtgaaagtggcagagatacttaactcaatggtaggtgccgcaccggggtacagagtcctcaacaggaacatcatcactgccgaagaggaagttctggtcaacgccgccaacagcaacggtagacccggtgacggtgtgtgtggtgcgctctacggcgcgtttggggacgcgttccccaacggtgcaatcggcgcgggaaacgcggtcctggttcgaggactcgaggctaccatcatacacgcagccggagctgacttcagagaggttgacgaagaaaccggcgcgcggcagctaagagcagcataccgcgcggcggctaccctagtcacggccaacggcatcaccagcgccgccatccccttgctgagtacacacatcttctctaacggtcggaacaggctggaacagtcgtttagtgcactggtggaggcgttcgacacgacagagtgcgacgtcaccatctactgcctcgccaacaacatggccgcgaggatccagcaactgattgacgcccacgctcgcgaagagtttgatgaggaagtggttgtggaagaggaagaggaacacgaagccgacgcaatgagtgacacggagacgctgtccagtttcggcgacgaaacggtatgggtgcctaaacacagcactctggctggaaggccagggtacagcgcctactacggcgaccgcagatccctttttgtcggcacgaaattccatcgtgcagcggtcgctatgtcgtcgattgaggcggcttggcctaagaccaaggaagccaatgctaaactcatcgagtatatacgagggcaacacctcgttgacgttctaaagagctgcccggttgacgacatacccgtgggcagaccgccttctagcctaccctgtggctgcatatacgccatgaccccggaacgggtcacagtactgaaacaaagaccgcaggaaggtttcgtggtgtgcagtgcattcaagctgcctctcaccaacatccaggacgtcaccaaggtggagtgcactgtgagagcacccgcagaggaacctagaccggtgcgccatctgcaggagaggcgcccagcgcaggccgccgtgaggcagcttaggccggcagccgtggctgccagtgtcgccgcgagtcacacagccagcaggaccagcactgcctcgagccgccgcacacctgcgccgggttcggtgcaggtgcgtttactgccgccgagagatggtacggaatcacgcagttctcgaatgggctcgcagtccagcgtcacatcatcagcgggatccgttccgccggcgccccgaagggcgccagctgtgtcggcggcatcgttggcgagcagcgcccatagccgcagtgtgcgcagcgcccctgctatgagggccgccagcgcgggtgccagaagcgtgcgcagcgcccaatccggttccaccgggcacagagcgggcgccttcagcgtcgccggctcggtgagacagcccagcgggccgcctagtagcgtgagcacacccgccgcgatcagagggctaacacgggaccagtttgacgccgtaagagttagggcccgcaggaacttggaacttgaggggtcggagcacggcagtcaatctagcttccactccggctcgctggcggtggggagctccgctagtagctatagccaacgctccgacgatcaggatacgggcactgaaccctcaagccgcggcgccgccgtgagaactcggcgcagagggcaacgggacggcctcggagggtacatattctcatctgatcaaggtacggctcacctcagccagcacaacacccagacaaacaacacaacggaagtgttgatgagaacgtcagtactgccgagcaacgaccacggcacaccagatctgcctgcagaaacgaggaagaggttggcgtatcaaatgcgccccactcaaaagaacaagagtcgctacctgtccgcgaaggtccataacatgaaacacaagattgttcgatgtttgcagcgaggtgccgggcactacctgagagagcagcacgctctgccgctgtggaaaaacaccttccctaagcccaggtactccgacgcctgcgtggtgaagttcgagagcgtcaacacagcaatcgttgcagccaacatgttcatagggtgtaattaccccaccttaagctcgttcggtatcacggataagtacgatgcttacctggacatggttgatgggctcaattgcaacctcgacaccgtgacgttcgaccctgctaaggtgcgttccctgccgaagaaatcagagtataaccagccgctcatacagtcccaagtacccggacctatgacatccactctgcaaagcatcctcatggccgccaccaaacgtaactgcaacgtcacgcagatgagagagctcccgactatggactcagcggcaatgaacgtggaggcttttaaaagtttcgcctgtaaggacaccgacctgtggactgagttcgcggaaaaaccagtaaggttgtcgcccggccaaatcgaagagtatgtctttcatctacaaggggccaaggccaatgtgatgcacagcagagtcgaagccgtatgccctgacctctcggaggtggctatggacaggttcacactagacatgaaacgcgacgtcaaagtgacgccaggcacgaagcacgtagaggagagacctaaagtccaagagattcaagcggccgaccccatggccaccgcgtacttgtgcgccatccatagagagctagtccgaaggctgaaggccgtcctgaaaccgtctatacacgtgttgttcgatatgagctccgaggattttgatgctatcgtgggccatgggatgaagttgggtgacaaggtgctggaaacggacatctcctcattcgacaagagccaggaccaagccatggcggttacagcgctgatgctgctgagggacttgggagtagaagaagacctcctgaccctaattgaggcgtctttcggcgacatcacttctgcccacctgcccacaggcaccagatttcagtttggatcgatgatgaagtctggactttttctgacgctgttcgtgaacacgctgcttaacatcaccatagctgcccgagttttacgggagcagctggctgataccaggtgtgccgcgtttatcggtgacgacaacgtaatcaccggagtagtgtctgacgacatgatggtggccaggtgcgcatcctggctgaacatggaggtgaagatcatggacatggaaattggcaacatgagtccttatttttgtggcggcttcctgttactcgacacggtaacaggcactgtaagccgagtgtcggaccctgtaaaacgcctgatgaagatgggaaaaccggccctgaacgatccagaaacggacgtggacagatgccgcgcactgcgcgaagaagtggaaagctggtacagagtggggattcagtggccactgcaggtggctgccgccacacgctatggcgtgaaccacctgccgctggccacaatggcgatggccacgctcgcccaggacttgagatcgtacctgggcgcgcgaggggagtacgtatccctctacgtctaaccttaatattttctgcatcatacttccaaacaatcatgtttcccatgcaattcaccaactcagcctatcgccagatggagcccatgtttgcaccgggttcccgaggacaagtacagccgtaccggccgcgcactaagcgccgccaggagccgcaagtcggcaacgccgccattactgccctcgcgaaccagatgagtgcgctccagttgcaggtagctggacttgccggccaggcaagggtggaccgccgtgggccaagacgtgttcagaagaacaagcagaagaagaagaactcttccaacggagaaaaacccaaagagaagaagaagaagcaaaaacaacaggagaagaagggaagcggtggcgaaaaagtcaagaagactaggaaccgacccgggaaggaggtaaggatctccgtaaagtgtgcccgacagagcaccttccccgtgtaccacgaaggtgctatatccggctacgctgtgctgattggatctcgcgtattcaagccggcacacgtgaagggtaagatcgaccaccctgaactggcagacatcaagttccaggtcgccgaggacatggacctcgaagcagctgcgtacccgaagagcatgcgagaccaagcggctgaaccagcgaccatgatggacagagtgtacaactgggagtatggcactatcagagtggaggataatgtcataatcgacgcaagcggtaggggcaagccgggtgacagtggcagggccatcaccgacaactcgggaaaggttgttggtattgtcctcggaggaggacccgatggcaggcgcacacgcctctccgtgataggtttcgacaagaagatgaaggctagggagatcgcctacagtgatgccataccttggacacgcgctccggccctcctgctgctgcctatggttattgtctgcacctacaattccaacaccttcgattgctccaaaccgtcctgccaggactgctgcattactgctgaaccagagaaggccatgaccatgctgaaggacaatctgaacgacccgaactactgggacctactcattgctgtcaccacctgtggctccgcccggagaaagagggctgtgtctacgtcgcctgccgccttttacgacacacagatcctcgccgcccacgcagctgcctccccatacagggcgtactgccccgattgtgacggaacagcgtgtatctcgccgatagccatcgacgaggtggtgagcagtggcagcgaccacgtcctccgcatgcgggttggttctcaatcgggagtgaccgctaagggtggtgcggcgggtgaaacctctctgcgatacctgggaagggacgggaaggttcacgccgcagacaacacgcgactcgtggtgcgcacgactgcaaagtgcgacgtgctgcaggccactggccactacatcctggccaactgcccagtggggcagagcctaaccgttgcggccacactggatggcacccggcatcaatgcaccacggttttcgaacaccaagtaacggagaagttcaccagagaacgcagcaagggccaccatctgtccgacatgaccaagaaatgcaccagattttccactacaccaaaaaagtccgccctctacctcgttgatgtgtatgacgctctgccgatttctgtagagattagcaccgtcgtaacatgcagcgacagccagtgcacagtgagggtgccacctggtaccacagtgaaattcgacaagaaatgcaagagcgctgactcggcaaccgtcactttcaccagcgactcccagacgtttacgtgtgaggagccagtcctaacggctgccagtatcacccagggcaagccacacctcagatcggcaatgttgcctagcggaggcaaggaagtgaaagcaaggatcccgttcccgttcccgccggaaaccgcaacttgcagagtgagtgtagccccactgccgtcgatcacctacgaggaaagcgatgtcctgctagccggtaccgcaaaataccctgtgctgctaaccacacggaaccttggtttccatagcaacgccacatccgaatggatccagggcaagtacctgcgccgcatcccggtcacgcctcaagggatcgagctaacatggggaaacaacgcgccgatgcacttttggtcatccgtcaggtacgcatccggggacgctgatgcgtacccctgggaacttctggtgtaccacaccaagcaccatccagagtacgcgtgggcgtttgtaggagttgcatgcggcctgctggctatcgcagcgtgcatgtttgcgtgcgcatgcagcagggtgcggtactctctggtcgccaacacgttcaactcgaacccaccaccattgaccgcactgactgcagcactgtgttgcataccaggggctcgcgcggaccaaccctacttggacatcattgcctacttgtggaccaacagcaaagtggccttcgggctacaatttgcggcgcccgtggcctgtgtgctcatcattacatacgcccttaggcactgcagattgtgctgcaagtcttttttaggggtaagagggtggtcagccctgctggtcatccttgcgtatgtacagagctgcaagagctacgaacacaccgtggtggtcccaatggatccaagagccccgtcgtacgaagcagtgataaaccggaatgggtatgatccattgaagctgaccatctcagtgaatttcaccgtcatctcaccaactacggctctggaatattggacctgcgcaggagtccccatcgtcgagccgccccatgtgggctgctgcacgtcggtgtcctgcccctctgacctctctacgctgcatgcgtttactggcaaagctgtctccgacgtgcactgcgatgtgcacacaaacgtgtaccccttgttgtggggcgcggctcactgcttctgttccaccgagaatacacaggtcagcgctgtggcagccaccgtttctgagttctgtgcccaggactcagagcgtgccgaagcgttcagcgtacacagcagctcagtcaccgctgaggtcctggtgacgcttggtgaagtggtgacggcagtccacgtttacgtggacggggtaacatcagccaggggcactgacctcaagatcgtggctggaccaataacaaccgactactccccattcgatcgcaaagtagtccgcatcggcgaagaggtctataactatgactggcctccttacggggctggccgaccaggcacattcggagacattcaagctaggtcaaccaactatgtcaaacccaacgatctgtatggggacatcggaattgaagtactgcagccgactaacgaccacgtacatgtggcttacacgtatacgacctctgggttactgcgttggctgcaggacgctccgaaaccactcagtgtcacagcaccgcacggttgtaagatcagtgccaatccgctcctggccctcgattgtggggttggtgccgtccccatgtccatcaacattccggacgcgaagtttacccgcaaattaaaggatccgaaaccatcggccctgaaatgcgtggtggacagctgcgagtacggggtggactacgggggcgccgccacgatcacctacgagggccacgaggccgggaagtgcgggattcattccctgacaccaggagtccccctgagaacatcggtggttgaagtggttgctggcgccaataccgtcaaaacgaccttctcctcacccacgcccgaggttgcactcgaggtagagatctgttcggcaatagtgaagtgcgctggtgagtgcactccaccgaaggaacatgtggtcgcaaccaggcctcgccatggcagcgaccctggaggctacatctccgggcccgcaatgcgctgggccggagggattgtagggaccctagtggtcctgttccttatccttgccgtcatctactgcgtggtgaagaagtgccgctccaaaagaatccggatagtcaagagctaaattccggtatacaaattgctcactaggagcccatccgatcccacagggagtaggatgagtcatctattggttttaaaattttcaatac

SEQ ID NO: 5 - DNA, 22 bp, synthetic construct
  misc_feature 1..22: note = Primer
  source 1..22: mol_type = other DNA
tattgccccgactgtgatggaa

SEQ ID NO: 6 - DNA, 19 bp, synthetic construct
  misc_feature 1..19: note = Primer
  source 1..19: mol_type = other DNA
aggatgtagtggccggtgg

SEQ ID NO: 7 - DNA, 20 bp, synthetic construct
  misc_feature 1..20: note = Primer
  source 1..20: mol_type = other DNA
aacgacaatcagtgcacagt

SEQ ID NO: 8 - DNA, 18 bp, synthetic construct
  misc_feature 1..18: note = Primer
  source 1..18: mol_type = other DNA
ggcattgctgtggaaacc

SEQ ID NO: 9 - DNA, 18 bp, synthetic construct
  misc_feature 1..18: note = Primer
  source 1..18: mol_type = other DNA
ggtttccacagcaatgcc

SEQ ID NO: 10 - DNA, 19 bp, synthetic construct
  misc_feature 1..19: note = Primer
  source 1..19: mol_type = other DNA
cgaccaaagagtaccgcac

SEQ ID NO: 11 - DNA, 18 bp, synthetic construct
  misc_feature 1..18: note = Primer
  source 1..18: mol_type = other DNA
gcgatacctgggaaggga

SEQ ID NO: 12 - DNA, 20 bp, synthetic construct
  misc_feature 1..20: note = Probe
  source 1..20: mol_type = other DNA
acaacacgcggcttgtggtg

SEQ ID NO: 13 - DNA, 18 bp, synthetic construct
  misc_feature 1..18: note = Primer
  source 1..18: mol_type = other DNA
tgcagcacatcgcacttt

SEQ ID NO: 14 - DNA, 20 bp, synthetic construct
  misc_feature 1..20: note = Primer
  source 1..20: mol_type = other DNA
ccaagcgaccgttaccttta

SEQ ID NO: 15 - DNA, 24 bp, synthetic construct
  misc_feature 1..24: note = Probe
  source 1..24: mol_type = other DNA
cagtttacgtgtgaggagccggtt

SEQ ID NO: 16 - DNA, 20 bp, synthetic construct
  misc_feature 1..20: note = Primer
  source 1..20: mol_type = other DNA
atgatctaaggtgcggcttg

SEQ ID NO: 17 - DNA, 20 bp, synthetic construct
  misc_feature 1..20: note = Primer
  source 1..20: mol_type = other DNA
cgtacccttgggaacttctg

SEQ ID NO: 18 - DNA, 24 bp, synthetic construct
  misc_feature 1..24: note = Probe
  source 1..24: mol_type = other DNA
cacaccaagcaccatccggagtac

SEQ ID NO: 19 - DNA, 18 bp, synthetic construct
  misc_feature 1..18: note = Primer
  source 1..18: mol_type = other DNA
agcagaccacatgcaact